(19) United States
(12) Patent Application Publication
(10) Pub. No.: US 2023/0414416 A1
(43) Pub. Date: Dec. 28, 2023

(54) ABSORBENT ARTICLES CONTAINING WETNESS INDICATING COMPOSITIONS AND METHODS FOR MANUFACTURE

(71) Applicant: The Procter & Gamble Company, Cincinnati, OH (US)

(72) Inventors: Nicholas David VETTER, Cleves, OH (US); Adrien GRENIER, Schwalbach (DE); Doris HOLLENBERG, Schwalbach (DE); Dirk SAEVECKE, Wiesbaden (DE)

(21) Appl. No.: 18/213,401

(22) Filed: Jun. 23, 2023

Related U.S. Application Data
(60) Provisional application No. 63/355,227, filed on Jun. 24, 2022.

(51) Int. Cl. A61F 13/42; A61F 13/84; A61L 15/56; C09B 49/12

(52) U.S. Cl. CPC A61F 13/42; A61F 13/84; A61L 15/56; C09B 49/12; A61F 2013/422; A61F 2013/8497; A61L 2300/216

(57) ABSTRACT

An absorbent article having a topsheet, a backsheet, an absorbent core disposed at least partially between the topsheet and the backsheet, and a wetness indicating composition is provided. The wetness indicating composition includes a colorant and a colorant fastener. The colorant has a first color state, wherein the first color state is associated with a first state of the wetness indicating composition. The colorant has a second, different color state, wherein the second color state is associated with a second state of the wetness indicating composition. The wetness indicating composition is disposed on the absorbent article in a plurality of discrete graphics, wherein each of the plurality of discrete graphics has an area of between about 0.02 cm² and about 25 cm².

### CROSS REFERENCE TO RELATED APPLICATIONS

This application claims the benefit, under 35 U.S.C. § 119(e), to U.S. Provisional Application No. 63/355,227, filed Jun. 24, 2022, which is incorporated by reference herein in its entirety.

### FIELD

The present disclosure is directed to absorbent articles, and methods of manufacturing absorbent articles, comprising wetness indicating compositions comprising a colorant and a colorant fastener.

### BACKGROUND

Absorbent articles are used in the hygiene industry to contain and absorb bodily exudates (i.e., urine, bowel movements, and menses) in infants, toddlers, children, and adults. Absorbent articles may include, but not be limited to, diapers, pants, adult incontinence products, feminine care products, and absorbent pads. Users of absorbent articles, such as wearers and caregivers, often desire to know whether and/or how much liquid is present in an absorbent article at a given time. For example, a parent or caretaker of an infant may desire to know if and/or how much urine is present in a diaper worn by the infant. In order to signal the presence of a liquid, or that a liquid came in contact with a portion of the absorbent article, the absorbent article may comprise a wetness indicator or plurality of wetness indicators.

One issue with wetness indicators is poor retention of certain components of the wetness indicator at the application site on the absorbent article upon contact with liquids. Attempts have been made to improve the liquid-fastness of the components of wetness indicators by incorporating the components into adhesive compositions having high melting temperatures. High melting temperature adhesives, however, can be difficult to apply, especially in small, relatively intricate designs. Wetness indicators using high melting temperature adhesives are typically applied to absorbent articles in large strips, requiring use of a relatively large amount of wetness indicating composition on each absorbent article. As such, absorbent articles comprising wetness indicating compositions should be improved.

### SUMMARY

Aspects of the present disclosure may solve one or more of the problems discussed above by providing an absorbent article comprising a wetness indicating composition comprising a colorant and a colorant fastener. The wetness indicating composition may be designed to facilitate application to an absorbent article or a component or substrate thereof in liquid form to allow for the creation of a plurality of discrete graphics using a relatively small amount of the wetness indicating composition.

The present disclosure provides, in part, an absorbent article comprising a topsheet, a backsheet, an absorbent core disposed at least partially between the topsheet and the backsheet, and a wetness indicating composition comprising a colorant and a colorant fastener. The colorant has a first color state, wherein the first color state is associated with a first state of the wetness indicating composition. The colorant has a second, different color state, wherein the second color state is associated with a second state of the wetness indicating composition. The wetness indicating composition may be disposed on the absorbent article in a plurality of discrete graphics, wherein each of the plurality of discrete graphics has an area of between about 0.02 cm² and about 25 cm², between about 0.035 cm² and about 15 cm², between about 0.05 cm² and about 10 cm², between about 0.05 cm² and about 5 cm², between about 0.065 cm² and about 2.5 cm², or between about 0.065 cm² and about 1.0 cm².

The present disclosure provides, in part, an absorbent article comprising a topsheet, a backsheet comprising a garment-facing surface and a wearer-facing surface, an absorbent core disposed at least partially between the topsheet and the backsheet, and a wetness indicating composition comprising a colorant and a colorant fastener. The colorant has a first color state, wherein the first color state is associated with a first state of the wetness indicating composition in which the colorant and the colorant fastener are not chemically bonded. The wetness indicating composition is in the first state prior to exposure of the wetness indicating composition to an activating liquid. The colorant has a second color state, wherein the second color state is associated with a second state of the wetness indicating composition in which at least a portion of the colorant is chemically bonded to at least a portion of the colorant. The wetness indicating composition is in the second state after exposure to the activating liquid. The wetness indicating composition may be substantially insoluble in the activating liquid when in the second state.

The present disclosure provides, in part, a method of making an absorbent article comprising a wetness indicating composition. The method comprises the steps of providing a substrate, providing a wetness indicating composition, applying the wetness indicating composition to the substrate by a printing process; and joining the substrate to at least one other component to form the absorbent article. The wetness indicating composition comprises a colorant, a solvent, and a colorant fastener. The colorant has a first color state, wherein the first color state is associated with a first state of the wetness indicating composition, and wherein the wetness indicating composition is in the first state prior to exposure of the wetness indicating composition to an activating liquid. The colorant has a second, different color state, wherein the second color state is associated with a second state of the wetness indicating composition, and wherein the wetness indicating composition is in the second state after exposure to the activating liquid. The wetness indicating composition is in a liquid state at 25° C.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of the absorbent article of FIG. 4, laid flat, with a garment-facing surface facing the viewer;

FIG. 7 is a cross-sectional view of the absorbent article taken about line 7-7 of FIG. 6;

FIG. 8 is a cross-sectional view of the absorbent article taken about line 8-8 of FIG. 6;

FIG. 9 is a plan view of an example absorbent core or an absorbent article;

FIG. 10 is a cross-sectional view, taken about line 10-10, of the absorbent core of FIG. 9;

FIG. 11 is a cross-sectional view, taken about line 11-11, of the absorbent core of FIG. 10;

FIG. 12 is a plan view of an example absorbent article of the present disclosure that is a sanitary napkin;

FIG. 13 is a plan view of an example absorbent article in the form of a taped diaper, garment-facing surface facing the viewer, in a flat laid-out state;

FIG. 14 is a plan view of a backsheet, wearer-facing side facing the viewer; and

FIG. 15 is a plan view of a backsheet containing a first plurality of graphics and a second plurality of graphics, wearer-facing side facing the viewer.

### DETAILED DESCRIPTION

Various non-limiting forms of the present disclosure will now be described to provide an overall understanding of the principles of the structure, function, manufacture, and use of the absorbent articles containing wetness indicating compositions disclosed herein. One or more examples of these non-limiting forms are illustrated in the accompanying drawings. Those of ordinary skill in the art will understand that the absorbent articles containing wetness indicating compositions and methods for manufacture described herein and illustrated in the accompanying drawings are non-limiting example forms. The features illustrated or described in connection with one non-limiting form may be combined with the features of other non-limiting forms. Such modifications and variations are intended to be included within the scope of the present disclosure.

### General Description of an Absorbent Article

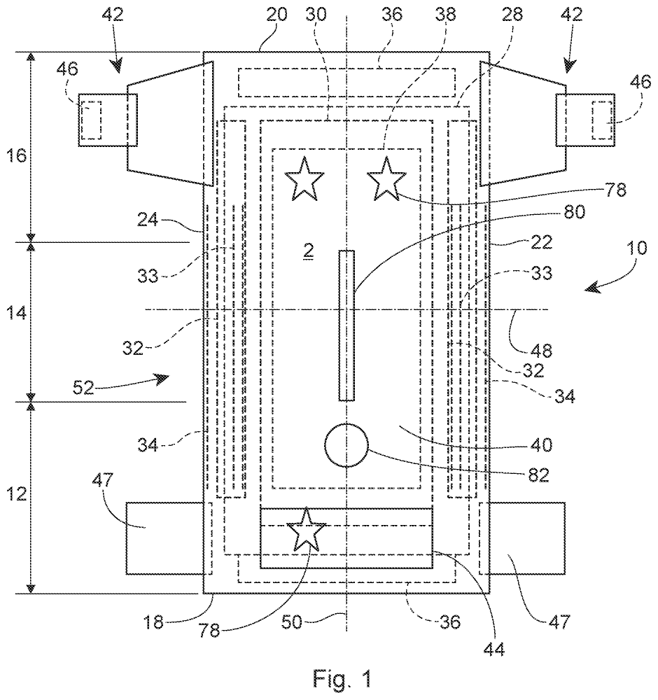

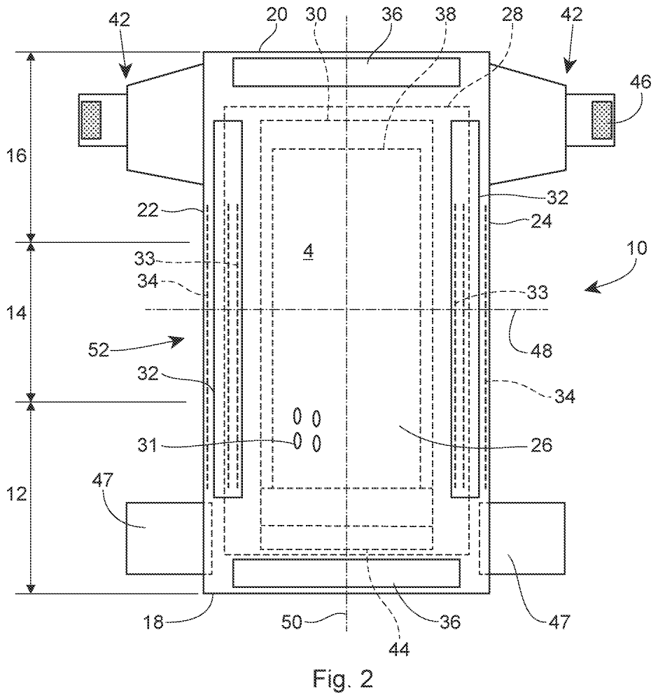

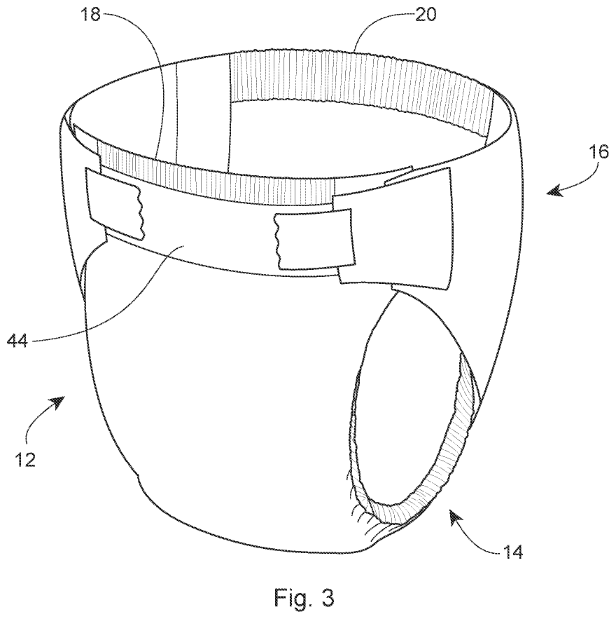

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be about ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50.

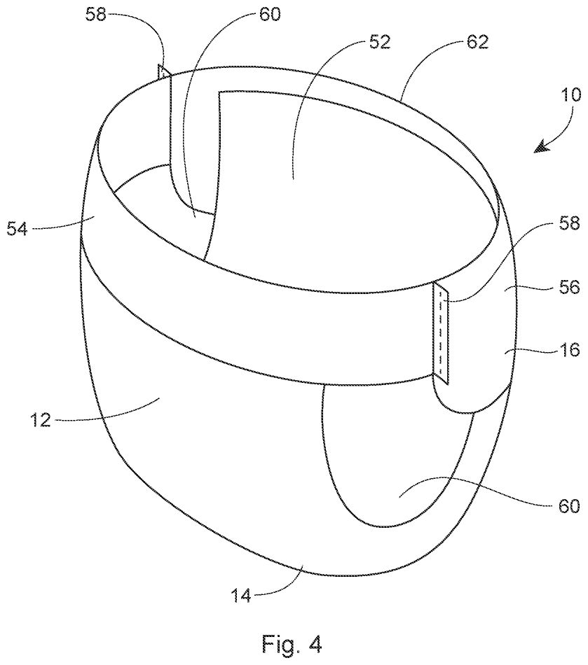

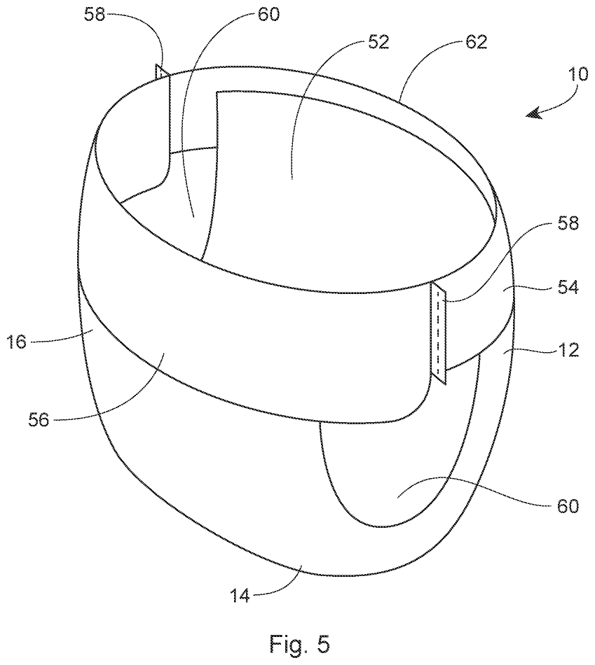

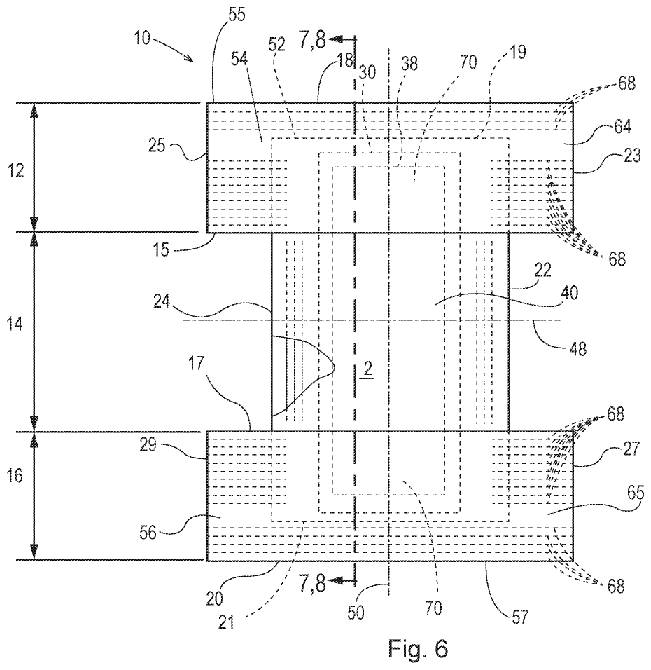

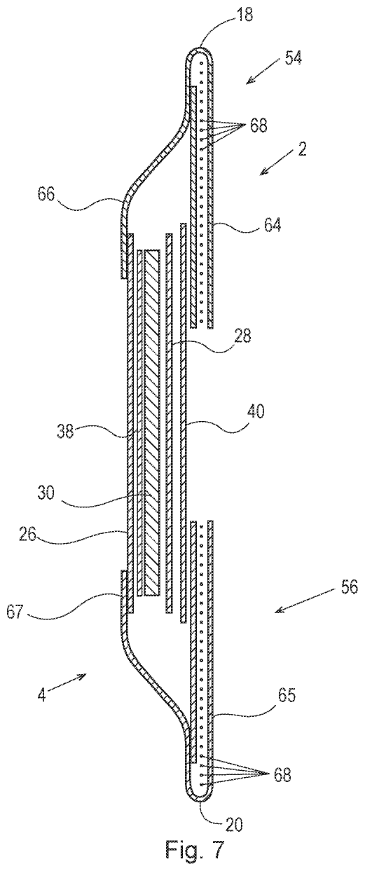

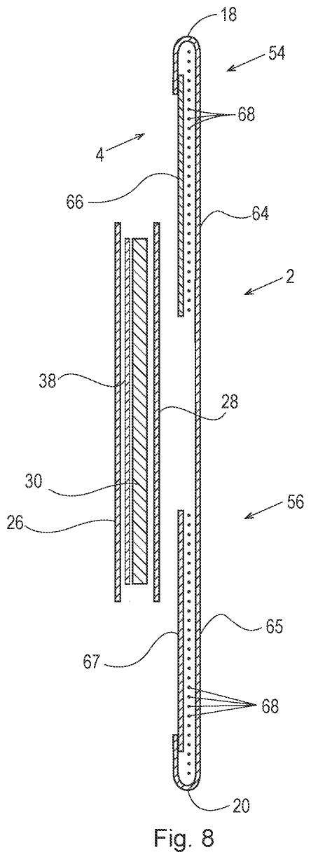

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137. Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. Appl. Pub. No. 2013/0211363.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt seams 15 and 17; or, alternatively, adjacent to the seams 58, 15, and 17 in the manner described in U.S. Pat. No. 9,498,389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). The topsheet may be apertured by overbonding a material and then rupturing the overbonds through ring rolling, such as disclosed in U.S. Pat. No. 5,628,097, to Benson et al., issued on May 13, 1997, and disclosed in U.S. Pat. Appl. Publication No. 2016/0136014 to Arora, et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material.

### Absorbent Core

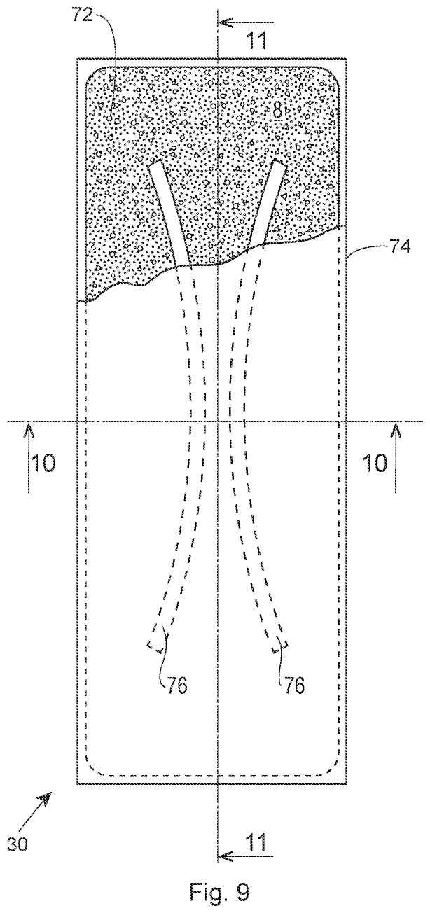

As used herein, the term “absorbent core” 30 refers to a component of the absorbent article 10 disposed in the article for absorbing and containing liquid such as urine received by the absorbent article. The absorbent core thus typically has a high absorbent capacity. An example absorbent core 30 is schematically shown in FIGS. 9-11. The absorbent core comprises an absorbent material 72, that is typically enclosed within or sandwiched between a core bag 74.

The core wrap may be a single material that is folded and attached to itself, or it may comprise a separate top layer and bottom layer that may be bonded or otherwise joined together. The absorbent material typically comprises superabsorbent particles which are optionally mixed with cellulose fibers. As used herein, “absorbent core” does not include any acquisition-distribution systems, topsheet, or backsheet of the absorbent article.

The example absorbent core 30 shown in isolation in FIGS. 9-11 is in the dry state (before use). The absorbent core may typically have a generally rectangular shape as defined by its longitudinal edges and transversal front edge and back edge or may have other shapes.

Absorbent material 72 may be deposited as an absorbent layer having a generally rectangular outline, as represented in FIG. 9. A wide variety of absorbent cores may also be used. The absorbent material 72 layer may also have a non-rectangular perimeter (“shaped” core), in particular, the absorbent material 72 may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “hourglass” for the area of the absorbent material.

The absorbent material 72 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores, where the absorbent material consists, or consists essentially, of SAP. The absorbent material may also be a high internal phase emulsion foam “Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may have a CRC value of at least 20 g/g, in particular of from 20 g/g to 40 g/g. “Superabsorbent polymer particles”, as used herein, refers to a superabsorbent polymer material which is in particulate form so as to be flowable in the dry state.

Various absorbent core designs comprising high amounts of SAP have been proposed in the past, see for example in U.S. Pat. No. 5,599,335 (Goldman), EP 1,447,066 (Busam), WO 95/11652 (Tanzer), U.S. Pat. Appl. Pub. No. 2008/0312622 A1 (Hundorf), WO 2012/052172 (Van Malderen). In particular, the SAP printing technology as disclosed in U.S. Pat. Appl. Pub. No. 2006/024433 (Blessing), U.S. Pat. Appl. Pub. No. 2008/0312617 and U.S. Pat. Appl. Pub. No. 2010/0051166 A1 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate such as a carded nonwoven layer, having a porous structure into which SAP particles have been deposited, may also be used in present disclosure.

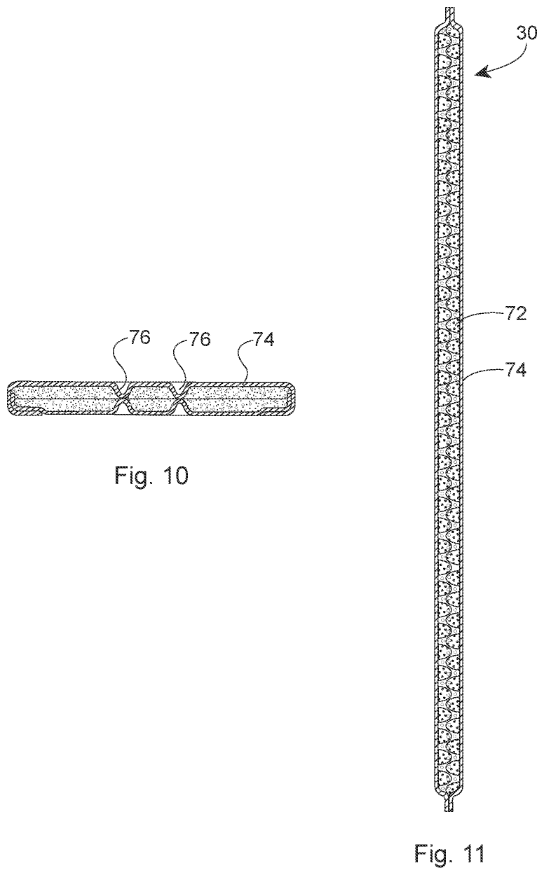

The absorbent material may be deposited as a continuous layer within the core wrap. The absorbent material may also be present discontinuously, for example, as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern, wherein the resulting layer is substantially continuously distributed across the absorbent particulate polymer material area, as illustrated in FIGS. 10-11. As for example taught in U.S. Pat. Appl. Pub. No. 2008/312,622 A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material within the absorbent material layer. The top side of the core wrap may be advantageously bonded to the bottom side of the core by adhesive, mechanical or ultra-sonic bonding through these material-free areas. Example disclosures of such channels in an airfelt-free core can be found in WO 2012/170778 (Rosati et al.) and US 2012/0312491 (Jackels). Channels may of course also be formed in absorbent cores comprising a mix of cellulose fibers and SAP particles. These channels may embody any suitable shapes and any suitable number of channels may be provided. In other instances, the absorbent core may be embossed to create the impression of channels. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4. As an example, a first elastic waistband 36 may be present in the front waist region 12 near the front belt end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, formed films, apertured formed films, cellulosic materials, cross-linked cellulosic materials, air laid cellulosic nonwoven materials, spunlace materials, or combinations thereof, for example. In some instances, portions of the acquisition materials 38 may extend through portions of the topsheet 26, portions of the topsheet 26 may extend through portions of the acquisition materials 38, and/or the topsheet 26 may be nested with the acquisition materials 38. Typically, an acquisition material 38 may have a width and length that are smaller than the width and length of the topsheet 26. The acquisition material may be a secondary topsheet in the feminine pad context. The acquisition material may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Graphics

Referring to FIG. 1, the absorbent articles 10 of the present disclosure may comprise graphics 78 that are visible from the garment-facing surface 2. The graphics 78 may be printed on the landing zone 44, the backsheet 28, and/or at other locations. The graphics 78 may coordinate with other elements of the absorbent article, such as bond patterns, wetness indicator(s) 80, aperture patterns, and the like.

### Front and Back Ears

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. The ears, or portions thereof, may be elastic or may have elastic panels. In an example, an elastic film or elastic strands may be positioned intermediate a first nonwoven material and a second nonwoven material. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 74 and the liquid impermeable backsheet 28.

### Sensors

Referring again to FIG. 1, the absorbent articles of the present disclosure may comprise a sensor system 82 for monitoring changes within the absorbent article 10. The sensor system 82 may be discrete from or integral with the absorbent article 10. The absorbent article 10 may comprise sensors that can sense various aspects of the absorbent article 10 associated with insults of bodily exudates such as urine and/or BM (e.g., the sensor system 82 may sense variations in temperature, humidity, presence of ammonia or urea, various vapor components of the exudates (urine and feces), changes in moisture vapor transmission through the absorbent articles garment-facing layer, changes in translucence of the garment-facing layer, and/or color changes through the garment-facing layer). Additionally, the sensor system 82 may sense components of urine, such as ammonia or urea and/or byproducts resulting from reactions of these components with the absorbent article 10. The sensor system 82 may sense byproducts that are produced when urine mixes with other components of the absorbent article 10 (e.g., adhesives, agm). The components or byproducts being sensed may be present as vapors that may pass through the garment-facing layer. It may also be desirable to place reactants in the absorbent article that change state (e.g., color, temperature) or create a measurable byproduct when mixed with urine or BM. The sensor system 82 may also sense changes in pH, pressure, odor, the presence of gas, blood, a chemical marker or a biological marker or combinations thereof. The sensor system 82 may have a component on or proximate to the absorbent article that transmits a signal to a receiver more distal from the absorbent article, such as an iPhone, for example. The receiver may output a result to communicate to the caregiver a condition of the absorbent article 10. In other instances, a receiver may not be provided, but instead the condition of the absorbent article 10 may be visually or audibly apparent from the sensor on the absorbent article.

### Packages

The absorbent articles of the present disclosure may be placed into packages. The packages may comprise polymeric films and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate number of absorbent articles per package. By packaging the absorbent articles under compression, caregivers can easily handle and store the packages, while also providing distribution savings to manufacturers owing to the size of the packages.

### Arrays

“Array” means a display of packages comprising disposable absorbent articles of different article constructions (e.g., different elastomeric materials [compositionally and/or structurally] in the side panels, side flaps and/or belts flaps, different graphic elements, different product structures, fasteners, or lack thereof). The packages may have the same brand and/or sub-brand and/or the same trademark registration and/or having been manufactured by or for a common manufacturer and the packages may be available at a common point of sale (e.g., oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” distributed by a common on-line source.

### Sanitary Napkin

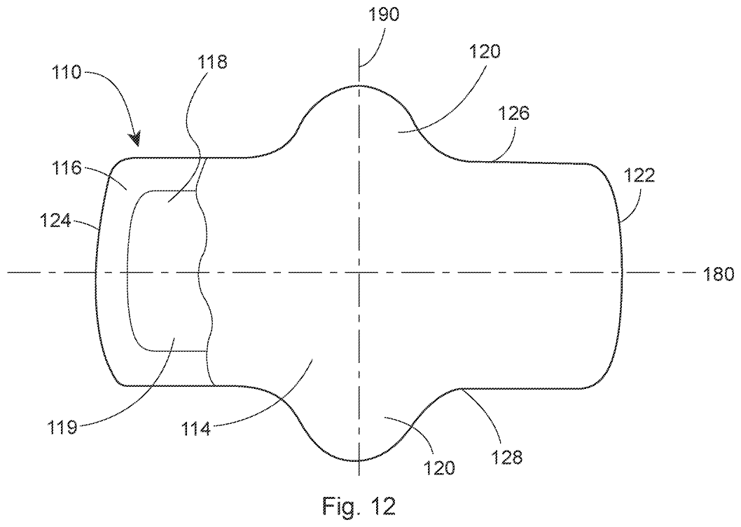

Referring to FIG. 12, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 128 to a midpoint of the second side edge 128. The sanitary napkin 110 may also be provided with additional features commonly found in sanitary napkins as is known in the art.

### Bio-Based Content for Components

Components of the absorbent articles described herein may at least partially be comprised of bio-based content as described in U.S. Pat. Appl. No. 2007/0219521 A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

### Wetness Indicator(s)

Users of absorbent articles, such as wearers and caregivers, often desire to know whether and/or how much liquid is present in an absorbent article at a given time. For example, a parent or caretaker of an infant may desire to know if and/or how much urine is present in a diaper worn by the infant. In order to signal the presence of a liquid, or that a liquid came in contact with a portion of the absorbent article, the absorbent article may comprise a wetness indicator or plurality of wetness indicators.

The wetness indicators of the present disclosure may comprise or consist of a wetness indicating composition. The wetness indicating compositions of the present disclosure comprise a colorant and a colorant fastener, which are illustrated in more detail herein. The wetness indicating compositions of the present disclosure may consist essentially of or consist of a colorant and a colorant fastener or may include additional ingredients. The wetness indicating compositions of the present disclosure may be disposed on, attached to, chemically bound to, or otherwise associated with a substrate—such as a structural component of an absorbent article or a substrate thereof.

The wetness indicating compositions of the present disclosure comprise a colorant. The colorant may have a first color state, wherein the first color state is associated with a first state of the wetness indicating composition. Examples of the first color state include, but are not limited to, colors visible to the human eye, such as red, blue, green, indigo, violet, yellow, orange, purple, black, and the like; colors not visible to the human eye, such as colors in the ultraviolet (UV) or infra-red (IR) portions of the electromagnetic spectrum, and the like. The first color state may also be translucent or opaque. The first color state of the wetness indicating compositions may be the same, or a similar, color as the substrate and/or component of the absorbent article on which it is applied. For example, the first color state of the wetness indicating compositions may be white and may be applied to a white-colored substrate or structural component of an absorbent article.

The colorant may have a second color state, wherein the second color state is associated with a second state of the wetness indicating composition. Examples of the second color state include, but are not limited to, colors visible to the human eye, such as red, blue, green, indigo, violet, yellow, orange, purple, black, and the like; colors not visible to the human eye, such as colors in the ultraviolet (UV) or infra-red (IR) portions of the electromagnetic spectrum, and the like. The second color state may also be translucent or opaque. The second color state is different than the first color state, having a change in intensity and/or visual distinctiveness. The first color state may be a first color, such as yellow, while the second color state may be a second color, such as blue; or the first color state may be a first color, such as red, while the second color state may be transparent. The change in color state from the first color state to the second color state may be visually observed (for example by a human, i.e., human-readable), or may be detected by a human (human-readable) or by a machine or device (machine-readable).

The wetness indicators of the present disclosure can provide a signal visible from outside the absorbent article while the absorbent article is being worn. The signal may be the change of the wetness indicating composition from the first color state to the second color state. The change in color state from the first color state to the second color state may be an irreversible, permanent color change. Thus, the wetness indicators of the present disclosure may be designed to have a wetness indicator state memory property, whereby after changing from the first color state to the second color state, the second color state is retained even upon discontinuation of exposure to an activating liquid or to conditions that are associated with the first state of the wetness indicating composition. Such a wetness indicator state memory property may be beneficial as a means to effectively record exposure of the wetness indicating compositions to activating liquids.

The first color state of the wetness indicating composition of the present disclosure is associated with a first state of the wetness indicating composition. This first state of the wetness indicating composition may include, but is not limited to: a specific pH or pH range; absence or presence of a specific compound or compounds, such as water, urea, dissolved oxygen, ions, such as, but not limited to, iron, calcium, sodium, chloride and combinations thereof, sugars, such as glucose, enzymes; and combinations thereof; microbiological flora and fauna, such as bacteria and the like; some threshold level of a compound or composition, such as water, urine, etc., below a certain amount; and combinations thereof.

The second color state of the wetness indicating composition of the present disclosure is associated with a second state of the wetness indicating composition. This second state of the wetness indicating composition may include, but is not limited to: a specific pH or pH range; absence or presence of a specific compound or compounds, such as water, urea, dissolved oxygen, ions, such as but not limited to, iron, calcium, sodium, chloride and combinations thereof, sugars, such as glucose, enzymes; and combinations thereof; microbiological flora and fauna, such as bacteria and the like; some threshold level of a compound or composition, such as water, urine, menses, blood and the like; and combinations thereof. The second state of the wetness indicating composition may be a specific pH range brought about by the presence of urine.

The first state of the wetness indicating composition of the present disclosure may be a specific pH or pH range, and the second state of the wetness indicating composition of the present disclosure may be a specific pH or pH range different to the specific pH or pH range of the first state. The second state may be the pH or pH range of urine, preferably human urine, as measured as a neat solution at human body temperature (typically about 37.6° C.). The pH or pH range of urine is typically about 5.5 to about 8.0. The first state may be a specific pH or pH range which is more acidic or more basic than the second state, that is, a pH of less than about 5.5 or greater than about 8.0. The colorant may be a pH indicator. Non-limiting examples of suitable pH indicators include: sulfonephthalein pH indicators, such as bromocresol green, m-cresol purple, cresol red, chlorophenol red, bromothymol blue, bromopyrogallol red, bromoxylenol blue, and bromophenol blue; monoazo dyes, such as acid alizarin violet N; monoazo pyrazolone dyes, such as acid yellow 34; diazo dyes, such as acid black 24; acid anthraquinone dyes, such as acid black 48; amphoteric anthraquinone dyes, such as acid blue 45; triphenylmethane dyes, such as acid fuchsin; phthalein type dyes, such as o-cresolphthalein; xanthene dyes, such as 2′,7′-dichlorofluorescein eosin B; heterocyclic acridine aromatics, such as acridine orange; diphenylmethane dyes, such as auramine 0; triphenylmethane dyes, such as basic fuchsin; cationic thiazine dyes, such as azure C; cationic anthraquinone dyes, such as basic blue 47; phthalocyanine type dyes, such as copper phthalocyanine; quaternized phthalocyanine type dyes, such as alcec blue; cationic polymethine dyes, such as astrazon orange G; anthraquinone type, such as alizarin; the neutral complex dyes, such as azure A eosinate; the terpene type dyes, such as trans-beta-carotene; and combinations thereof.

The colorant of the present disclosure may be a sulfonephthalein pH indicator, such as, but not limited to, bromocresol green, m-cresol purple, cresol red, chlorophenol red, bromothymol blue, bromopyrogallol red, bromoxylenol blue, bromophenol blue, and combinations thereof. In an acidic state, the sulfonephthalein class of indicators have a neutral charge and are typically yellow in color. Upon contact with an activating liquid, such as urine, having a pH higher than their pKₐ, the sulfonephthalein class of pH indicators typically change to a blue-green color and acquires an anionic charge—which may allow the colorant to then be scavenged by a cationic compound. Bromophenol blue, for example, may change from a yellow (first color state) neutral state (first state of the wetness indicating composition) to a blue (second color state) anionic state (second state of the wetness indicating composition) at a pH greater than its pKₐ of approximately 4.0. Bromocresol green may change from a yellow (first color state) neutral state (first state of the wetness indicating composition) to a blue (second color state) anionic state (second state of the wetness indicating composition) at a pH greater than its pKₐ of approximately 4.7.

The wetness indicating composition of the present disclosure may comprise two or more colorants, each having at least one of their first and second state different, i.e., different pKₐ values, a pH, an enzyme trigger, etc., colors, or other properties. The varying first and second states may facilitate interactive scenes, sequences, or displays providing information regarding relative fullness/wetness of the article, or merely provide entertainment and/or aesthetic value. For example, the wetness indicating composition may contain one colorant that turns blue and another that turns red upon contact with an activating liquid. Alternatively, one portion of the graphic may appear, and another portion may disappear upon contact with an activating liquid, such as urine, menses, blood, and the like. In another form, the wetness indicating composition may comprise two or more colorants, each having their first and/or second state the same.

Without wishing to be bound by theory, it is believed that when a colorant of the present disclosure comes into contact with an activating liquid, such as urine, the charge of the colorant may change from neutral to anionic, not only effecting a color change in the colorant, but also causing the colorant to become more susceptible to associating with polar compounds—such as water and urine. This may lead to the increased solubility of the colorant in the activating liquid and an undesired removal from the absorbent article. In order to reduce or prevent this undesirable reaction, the wetness indicating composition of the present disclosure may comprise a colorant fastener.

The colorant fastener of the present disclosure may be any material which immobilizes the colorant when the colorant is in the second color state. The colorant fastener may comprise a cationic molecule, such as a permanently charged cationic molecule, which may associate with the second color state of the colorant to reduce or completely inhibit the solubility of the colorant in the activating liquid, or any other liquid. Thus, the colorant fastener may be particularly beneficial in “locking” the colorant within the wetness indicating composition and/or to a component of the absorbent article, such as the backsheet for example. Therefore, the wetness indicating compositions of the present disclosure may be less able to diffuse away from the application area and through the absorbent article when in contact with an activating liquid.

The colorant fastener of the present disclosure may comprise or consist of a quaternary ammonium compound. Without wishing to be bound by theory, it is believed that the cationic quaternary ammonium compound may form an ionically bonded coacervate with the anionic pH-sensitive colorant due to the strong coulombic interactions between the anionic colorant and the cationic quaternary ammonium compound. The coacervate complex formed between the anionic colorant and the cationic quaternary ammonium compound may neutralize the charge in both species and dramatically reduce both of their solubilities in polar solvents such as water or urine, thereby significantly reducing colorant leaching and improving colorant fastening to the absorbent article and/or portions thereof. Intermolecular forces between the coacervate and the absorbent article and/or portions thereof may further limit diffusion and/or mobility of the colorant (and wetness indicating composition) into an aqueous environment such as urine.

Quaternary ammonium compounds and salts thereof useful in the present disclosure includes those having the following formula (Formula I):

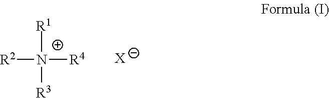

wherein each of R¹, R², R³ and R⁴ is independently selected from an alkyl group of from 1 to 30 carbon atoms or an aromatic, alkoxy, polyoxyalkylene, alkylamido, hydroxyalkyl, aryl or alkylaryl group having up to about 30 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated.

Dialkyl dimethyl ammonium quaternary compounds may be effective as colorant fasteners in wetness indicating compositions of the present disclosure. Dialkyl dimethyl ammonium quaternary compounds include, but are not limited to, dicocoalkyl dimethyl ammonium chloride, di(hydrogenated tallowalkyl) dimethyl ammonium chloride, distearyl dimethyl ammonium chloride, and (2-ethylhexyl)(hydrogenated tallowaklyl) dimethyl ammonium sulfate. Alkyl trimethyl ammonium compounds may also be effective as colorant fasteners in the wetness indicating compositions of the present disclosure, including dodecyl trimethyl ammonium chloride, hexadecyl trimethyl ammonium methylsulfate, and octadecyl trimethyl ammonium chloride. Other classes of cationic quaternary ammonium salts that may be colorant fasteners can include imidazoline quaternary class, the mono-, di-, tri-, and tetra-amidoamine quaternary classes, the mono-, di-, tri-, and tetra-benzyl quaternary classes, the benzlalkyl quaternary class of cationic compounds, the cationic diquaternary class, and the cationic ethoxylated quaternary class. It should be noted that the counter anion used in neutralization of the cationic quaternary compound may not be specifically limited to chloride. Other anions may also be employed, and non-limiting examples include bromide, methyl sulfate, and nitrate.

Mono-, di-, tri-, and tetra-alkyl classes of quaternary ammonium compounds may be especially effective in reducing or eliminating solubilization and/or diffusion of anionic colorants from the location of application on the absorbent article or components/substrates thereof. Compounds having a greater number of carbon atoms may be preferable for use in the wetness indicating composition of the present disclosure, and specifically for printable ink formulations, due to their compatibility with both the colorant and the solvent(s), and more specifically the volatile solvent(s), that may also be present in the wetness indicating composition, as discussed further herein. Such compounds having a greater number of carbon atoms may include tetraoctyl ammonium bromide, didocecyl dimethyl ammonium chloride, dimethyl dioctadecyl ammonium chloride, tetraoctyl ammonium bromide, tetrapentyl ammonium chloride, tetrabutyl ammonium chloride, hexadecyl trimethyl ammonium chloride, and the like.

Other cationic materials that may be useful aids in the inhibition of anionic colorant dispersion may include amine acid salts, polyacramidopropyltrimonium chloride, PEG-2 dimeadowfoamamidoethylmonium methosulfate, meadowfoam glyceryl quaternium, alkyl betaines, alkyl amido betaines, imidazolinium betaines, sulfobetaines, quaternized poly(vinylpyridine), amidoamine acid salts, poly(imine) acid-salts, polyethylene imine acid salts, cationic polyacryamides, poly(vinylamine) acid salts, cationic ionene polmers, poly(vinylbenzyl onium salts), poly(vinylimidazolinium salts), quaternized silicone compounds such as the diquaternary polydimethylsiloxanes, poly(vinyl alcohol) quaternary materials, cationic guars, polydimethyl diallylammonium chloride, cationic and anionic exchange resins and polymers, copolymers of vinylpyrrolidone and methylacrylamidopropyltrimethylammonium chloride, acidified polyvinylpyrrolidones, acidified polyvinylpolypyrrolidones, acidified copolymers of vinylpyrrolidone and vinylacetate, acidified copolymers of vinylpyrrolidone and dimethylaminoethylmetacrylate, copolymers of vinylpyrrolidone and methacrylamidopropyl trimethylammonium chloride, copolymers of quaternized vinylpyrrolidone and dimethylaminoethylmethacrlate, acidified vinylcaprolactam based polymers, acidified copolymers of vinylpyrrolidone and styrene, acidified copolymers of vinylpyrrolidone and acrylic acid, cationic polyelectrolyte polymers, and acidified n-alkyl-2-pyrrolidones.

It has been found that a relatively low molar ratio of quaternary ammonium compound to colorant (colorant fastener:colorant) may be particularly effective to reduce or eliminate diffusion of the colorant from the absorbent article or components thereof while reducing or preventing premature activation of the colorant. Quaternary ammonium compounds that may be useful as colorant fasteners may also exhibit hygroscopic properties and may draw moisture from the atmosphere, causing the wetness indicating composition to prematurely activate. Therefore, it may be desirable to limit the ratio of quaternary ammonium compounds to colorant in order to provide the colorant fastening benefit while limiting or preventing the potential for premature activation of the colorant. The wetness indicating composition of the present disclosure may have a molar ratio of colorant fastener to colorant of between about 1:1 and about 3:1, between about 1:1 and about 2.65:1, between about 1:1 and about 2.3:1, between about 1:1 and about 2:1, or between about 1:1 and about 1.75:1, specifically reciting all values within these ranges and any ranges formed therein or thereby. The wetness indicating composition of the present disclosure may have a molar ratio of colorant fastener to colorant of less than 15:1, less than 12:1, less than 10:1, less than 8:1, or less than 5:1. Wetness indicating compositions within the above-described molar ratios of colorant fastener to colorant may exhibit suitable colorant fastening properties while being suitably resistant to premature colorant activation. In a form, the absorbent articles of the present disclosure may comprise a topsheet, a backsheet, an absorbent core disposed at least partially between the topsheet and the backsheet, and a wetness indicating composition comprising a colorant and a colorant fastener; wherein the colorant has a first color state, wherein the first color state is associated with a first state of the wetness indicating composition; wherein the colorant has a second, different color state, wherein the second color state is associated with a second state of the wetness indicating composition; wherein the wetness indicating composition has molar ratio of colorant fastener to colorant of between about 1:1 and about 3:1, between about 1:1 and about 2.65:1, between about 1:1 and about 2.3:1, between about 1:1 and about 2:1, or between about 1:1 and about 1.75:1; and wherein the wetness indicating composition is disposed on the absorbent article in a plurality of discrete graphics, wherein at least a portion of individual graphics of the plurality of discrete graphics has an area of between about 0.02 cm² and about 25 cm². In another form, the absorbent articles of the present disclosure may comprise a topsheet, a backsheet comprising a garment-facing surface and a wearer-facing surface, an absorbent core disposed at least partially between the topsheet and the backsheet, and a wetness indicating composition comprising a colorant and a colorant fastener; wherein the colorant has a first color state, wherein the first color state is associated with a first state of the wetness indicating composition in which the colorant and the colorant fastener are not chemically bonded, and wherein the wetness indicating composition is in the first state prior to exposure of the wetness indicating composition to an activating liquid; wherein the colorant has a second color state, wherein the second color state is associated with a second state of the wetness indicating composition in which at least a portion of the colorant is chemically bonded to at least a portion of the colorant fastener, and wherein the wetness indicating composition is in the second state after exposure to the activating liquid; wherein the wetness indicating composition has molar ratio of colorant fastener to colorant of between about 1:1 and about 3:1, between about 1:1 and about 2.65:1, between about 1:1 and about 2.3:1, between about 1:1 and about 2:1, or between about 1:1 and about 1.75:1; and wherein the wetness indicating composition is substantially insoluble in the activating liquid when in the second state.

As discussed herein, the colorant of the present disclosure may have a first color state associated with the first state of the wetness indicating composition, and a second color state associated with the second state of the wetness indicating composition. It is believed that, when a colorant of the present disclosure is in the first color state, the colorant may not be chemically bonded to the colorant fastener. It is also believed that, when a colorant of the present disclosure is in the second color state, at least a portion of the colorant may be chemically bonded to at least a portion of the colorant fastener. At least a portion of the colorant fastener may be chemically bonded to at least a portion of the colorant in the second color state when the wetness indicating composition is in a second state by one or more selected from the group consisting of covalent bonding, ionic bonding, and combinations thereof. The resulting coacervate may be less soluble or substantially insoluble in an activating liquid and/or hydrophobic. The resulting coacervate may also have increased intermolecular forces between the coacervate and other components of the wetness indicating composition, further limiting diffusion and mobility of the colorant into liquids, such as the activating liquid.

By way of example, when the colorant is a pH-sensitive compound and the first state of the wetness indicating composition is a low pH, the colorant may have a neutral charge—such as in the case of the sulfonephthalein class of pH indicators—and may therefore not be chemically bonded to a colorant fastener because of the lack of ionic attractive forces in the first state. When the colorant is in the second color state and associated with a second state of the wetness indicating composition that is a higher pH (above the pKₐ of the colorant), the at least a portion of the colorant may have a negative charge (anion), and at least a portion of the colorant may be chemically bonded to at least a portion of the colorant fastener, at least a portion of which has a positive charge (cation). It is believed that at least a portion of the anionic colorant may form an ionically bonded coacervate with at least a portion of the cationic colorant fastener.

In one embodiment, the colorant may be bromocresol green, and may have a first color state associated with a first state of the wetness indicating composition, wherein the first state is a pH in the range of about 2 to about 4.5, and wherein the colorant has a neutral charge. The colorant having a first color state is not chemically bonded to a colorant fastener. The bromocresol green colorant may have a second, different color state associated with a second state of the wetness indicating composition, wherein the second state is a pH in the range of about 4.6 to about 8.5, and wherein the colorant has a negative charge. At least a portion of the bromocresol green colorant having a second color state may be chemically bonded to at least a portion of the colorant fastener. The colorant fastener may be a quaternary ammonium compound having a positive charge in the second state of the wetness indicating composition.

The wetness indicating compositions of the present disclosure may comprise one or more solvents in which the colorant, the colorant fastener, and any additional components may be solubilized prior to application to an absorbent article or a component or substrate thereof. The solvent(s) may be a volatile solvent, designed to evaporate once applied to an absorbent article of a component thereof. The solvent(s) may be a liquid (in a liquid state) at 25° C. The solvent(s) may be selected to solubilize or dissolve the other components of the wetness indicating composition and maintain the wetness indicating composition in a liquid state at about 25° C. (room temperature). Solvents of the present disclosure may comprise or consist of alcohols, such as 1-propanol, methanol, ethanol, isopropanol, butanol, isobutanol, and the like; acetates, such as propyl acetate, ethyl acetate, glycol ethers, propylene glycol, diethylene glycol, triethylene glycol, tripropylene glycol, and the like, and combinations thereof.

The wetness indicating compositions of the present disclosure may be liquid (in a liquid state) at room temperature (approx. 25° C.) prior to application to an absorbent article or a component thereof. The wetness indicating composition may have a viscosity of less than 200 cP, preferably less than 100 cP, preferably less than 75 cP, as measured at 25 C. The wetness indicating compositions of the present disclosure may comprise viscosity modifiers such as organoclays, paraffin waxes, micronized polytetrafluoroethylene (PTFE), cellulosic rheology inhibitors (such as methyl cellulose, hydroxypropyl methyl cellulose, etc.), melamine/formaldehyde capsules, urea/formaldehyde capsules, silica (SiO₂) (such as fumed silica, hydrophilic fumed silica, chemically modified silica, and combinations thereof. In addition to increasing viscosity, it has been found that the inclusion of silica in the printable ink formulation can impart a matte finish to the printed ink and/or reduce the stickiness/tackiness of the printed ink.

It has also been found that silica may affect the rate of the color change reaction. In particular, it has been found that the ratio of resin:silica may affect the rate of the color change reaction. As an illustrative example including a polyketone resin and hydrophilic fumed silica, a higher resin:silica ratio (such as >4:1) may provide a slower color change reaction rate, whereas a lower resin:silica ratio (such as <4:1) may provide a faster color change reaction rate. As to particular embodiments including a substrate comprising low-density polyethylene, when a relatively fast color change reaction rate (for example, wherein the color changes in <2 minutes) is desired, a resin (for example, a polyketone resin):silica (for example, hydrophilic fumed silica) ratio of about 2.6:1 may result in wetness indicating composition having such a relatively fast reaction rate. Where the substrate comprises a nonwoven material, a wetness indicating composition having a resin (for example, a polyketone resin):silica (for example, hydrophilic fumed silica) ratio of about 1.8:1 may result in a relatively fast color change reaction rate of, for example, less than 2 minutes.

More generally, when a color change reaction time of not greater than about 2 minutes is desired, a resin:silica ratio of between about 1:1 and about 2:1 may be useful with the present composition. When a color change reaction time of between about 2 and about 4 minutes is desired, a resin:silica ratio of between about 2.5:1 and about 3.5:1 may be useful with the present composition. When a color change reaction time of not less than about 15 minutes is desired, a resin:silica ratio of not less than about 4:1 may be useful with the present composition.

Absorbent articles of the present disclosure may comprise wetness indicating compositions comprising a colorant and a colorant fastener; wherein the colorant has a first color state, wherein the first color state is associated with a first state of the wetness indicating composition; wherein the colorant has a second, different color state, wherein the second color state is associated with a second state of the wetness indicating composition; wherein the colorant changes from the first color state to the second color state upon exposure of the wetness indicating composition to an activating liquid; and wherein the change from the first color state to the second color state occurs within 1 minute, within 2 minutes, within 3 minutes, within 4 minutes, or within 5 minutes of exposure of the wetness indicating composition to the activating liquid.

Of note, it has been found that in accordance with the present invention, as to particular embodiments, the inclusion of silica in the present composition can affect the printed surface, such as by increasing the texture/surface area of the substrate (due to the higher solids content) which, as detailed above, may facilitate an increased (i) rate of the color change reaction and/or (ii) color density. Further, the inclusion of silica in the present composition can increase the hydrophilicity thereof, which may also facilitate an increased rate of the color change reaction.

The wetness indicating compositions of the present disclosure may have a Glass Transition Temperature (Tg) of less than 150° C., preferably less than 100° C. Due at least in part to exhibiting the melt temperature, viscosity, and/or glass transition properties discussed herein, the wetness indicating compositions of the present disclosure may be configured to be applied to an absorbent article or a component thereof by various printing techniques. The wetness indicating composition of the present disclosure may be applied to an absorbent article or a component or substrate thereof by a flexographic printing process, a gravure printing process, a digital printing process, such as an ink jet printing process, and combinations thereof. The wetness indicating composition of the present disclosure may be configured to be printed onto an absorbent article or a component or substrate thereof by a high speed flexographic process with a line speed of at least about 1,000 feet/minute, at least about 1,300 feet/minute, at least about 1,500 feet/minute, or between about 1,000 feet/minute and about 5,000 feet/minute, between about 1,000 feet/minute and about 4,000 feet/minute, or between about 1,200 feet/minute and about 3,500 feet/minute, specifically reciting all values within these ranges and any ranges formed therein or thereby.

Because the wetness indicating compositions of the present disclosure may be liquid at room temperature and/or may possess the viscosity and/or glass transition properties discussed herein and therefore can be applied by a printing process, as opposed to, for example, a slot coating process (as is used with wetness indicating compositions having higher viscosities, glass transition temperatures, and melt temperatures), the wetness indicating composition may be disposed on the absorbent article or a component or substrate thereof in a plurality of discrete graphics, wherein each of the plurality of discrete graphics may be relatively small and/or intricate and/or disposed in distinct patterns. As used herein, the term “graphic” or “graphics” refers to the depiction of a design or designs, any recognizable indicia, such as a number, letter, word, brand name, icon, logo, character, any shape, or symbol (for example stripes, dots, geometric shapes, hearts, clouds, and individual elements that make up a larger design). In addition, because the wetness indicating compositions of the present disclosure may be less prone to diffuse away from the application area and through the absorbent article when in contact with an activating liquid, a clearer, more distinct, and/or more intricate wetness indicator pattern may be produced as compared to wetness indicating compositions that do not comprise a colorant fastener, because wetness indicating compositions that diffuse out of the wetness indicator pattern tend to appear blurred and less distinct. As opposed to large strips of wetness indicating composition, the wetness indicating composition of the present disclosure may be disposed on an absorbent article in a plurality of discrete graphics, wherein at least a portion of each of the plurality of discrete graphics has an area of between about 0.02 cm² and about 25 cm², between about 0.035 cm² and about 15 cm², between about 0.05 cm² and about 10 cm², between about 0.05 cm² and about 5 cm², between about 0.065 cm² and about 2.5 cm², or between about 0.065 cm² and about 1.0 cm², specifically reciting all values within these ranges and any ranges formed therein or thereby. At least a portion of each individual graphic of the plurality of discrete graphics may be spaced apart from others of the plurality of discrete graphics by at least 2 mm, at least 5 mm, at least 8 mm, at least 12 mm, or at least 15 mm. Such a plurality of graphics and dispersed disposition of the wetness indicating compositions of the present disclosure may not only be more aesthetically pleasing as compared to a single strip of wetness indicating composition as is typical of hot-melt formulations, but also may improve the functioning of the absorbent article. By disposing a plurality of discrete graphics over a portion of the absorbent article, the article may be able to detect wetness over a greater area of the article and notify a user or caretaker earlier of a wetness incident. And, in part, because of the relatively low viscosity and thin application of the wetness indicating composition of the present disclosure, a greater area of the absorbent may be covered by the same amount, or less, wetness indicating composition. In contrast, where an absorbent article has a wetness indicating composition disposed in a single strip—such as when the wetness indicating composition is in the form of a hot-melt adhesive—wetness will only be indicated when the wetness reaches the single strip of wetness indicating composition. The wetness indicating composition of the present disclosure may be disposed on an absorbent article in a plurality of at least 5, at least 8, at least 10, at least 15, at least 25, or at least 50 individual, discrete graphics.

Furthermore, in contrast to other wetness indicating compositions—especially hot-melt adhesive-based wetness indicating compositions—the wetness indicating composition of the present disclosure may be incorporated into an absorbent article at a relatively low level. An absorbent article of the present disclosure may comprise no more than 50 mg, no more than 35 mg, no more than 25 mg, no more than 15 mg, no more than 10 mg, or between about 3 mg and about 50 mg, between about 1 mg and about 35 mg, between about 1 mg and about 25 mg, between about 1 mg and about 15 mg, between about 1 mg and about 10 mg of the wetness indicating composition, specifically reciting all values within these ranges and any ranges formed therein or thereby. While the application level of the wetness indicating composition may be relatively low, the application area may be relatively extensive. An absorbent article of the present disclosure may comprise the wetness indicating composition of the present disclosure, wherein the wetness indicating composition is disposed on between about 0.5% and about 65%, between about 1% and about 50%, between about 5% and about 40%, or between about 8% and about 38% of a surface of a component or substrate of the absorbent article, specifically reciting all values within these ranges and any ranges formed therein or thereby. The relatively low application level and relatively high application area of the wetness indicating composition may therefore result in the wetness indicating composition having a relatively small thickness (as measured from the surface of the absorbent article or a component or substrate thereof in the z-direction) of between about 0.5 μm to about 12 μm, between about 1 μm to about 10 μm, or between about 4 μm to about 8 μm, specifically reciting all values within these ranges and any ranges formed therein or thereby, which may be quite different as compared to hot-melt-based wetness indicating compositions. The extensive application area of the wetness indicating composition of the present disclosure may improve the wetness-indicating function of the absorbent article, as wetness may be detected over a much broader area of the absorbent article rather than in just a central area.

The wetness indicating compositions of the present disclosure may comprise additional ingredients. The wetness indicating compositions may comprise resins such as polyvinyl butyrals (such as MOWITAL BA 20S, available from Kuraray Co., Ltd.), polyketone resins (such as REACTOL 1717H, available from Lawter, Inc), polyamide resins (such as FLEX-REZ 1155AS, available from Lawter, Inc.), phenolic resins (such as REACTOL 1111E, available from Lawter, Inc.), fumaric resin esters (such as FILTREZ 526 A, available from Lawter, Inc.), polyurethane resins (such as NEOREZ U-335, available from DSM Coating Resins B.V.), nitrocellulose resins, and the like. The resin(s) may have a Glass Transition Temperature (Tg) of less than 150° C., preferably less than 100° C. A resin having a Glass Transition Temperature of less than 150° C., preferably less than 100° C. is believed to be beneficial to the wetness indicating composition because it allows for adhesion to a substrate while also permitting flexibility to the applied (dried) wetness indicator, reducing or preventing cracking and chipping of the wetness indicating composition, and/or allows the resin to be incorporated into a wetness indicating composition that is configured to be printed, and therefore is liquid at room temperature.

The wetness indicating compositions of the present disclosure may comprise one or more acids, which may stabilize the colorant in a first color state and reduce or prevent premature color changes. Acids may include organic acids, such as, but not limited to, fatty acids such as stearic acid, palmitic acid, fruit acids e.g., citric acid, and salicylic acid; esters, such as, citrate esters, e.g., monostearyl citrate, glycolate esters, lactate esters; phosphorus containing organic acids, such as, monostearyl phosphate; ether carboxylic acids; N-acyl sarcosinic acids; N-acyl glutamic acids; N-acyl ethylenediaminetriacetic acid; alkane sulfonic acids; alpha-olefin sulfonic acids; alpha-sulfonic acid fatty acid methyl esters; sulfate esters; inorganic acids, such as, phosphoric acid; and combinations thereof.

The wetness indicating composition of the present disclosure may be adjustable for drying rate and/or press/printing runability, for example by including a solvent(s) which can slow down the evaporation rate, or by adding a solvent(s) which can speed up the evaporation rate—for example evaporation of the solvent after application to the absorbent article or component or substrate thereof.

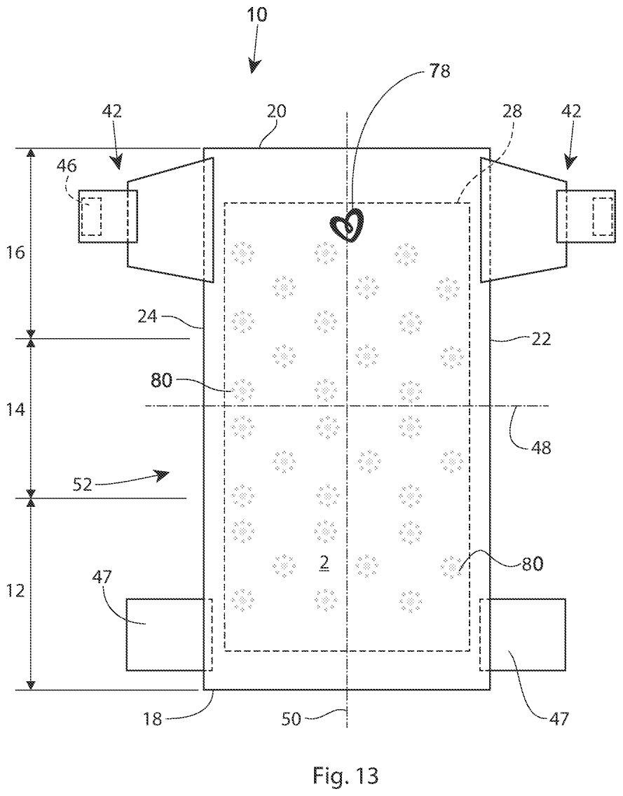

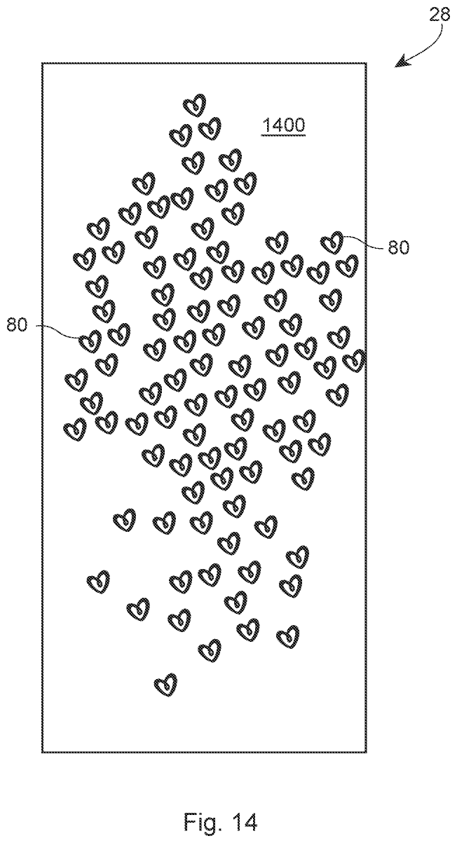

Referring to FIG. 13, the absorbent articles 10 of the present disclosure may comprise wetness indicators 80 comprising a wetness indicating composition of the present disclosure that may be visible from the garment-facing surface 2 of the absorbent articles 10. The wetness indicators 80 may be disposed on the landing zone 44, the backsheet 28, and/or at other locations. The wetness indicators 80 may be disposed on the absorbent core facing side 1400 of the backsheet 28, so that they can be contacted by bodily exudates within the absorbent core 30. As shown in FIG. 14, the wetness indicators 80 may be disposed only on a first surface 1400 of the backsheet 28, for example, only on the absorbent core-facing (i.e., wearer-facing) surface of the backsheet 28.

In some instances, the wetness indicators 80 may coordinate with graphics 78. For example, a wetness indicator may appear or disappear and create/remove a character in coordination with graphics 78. In other instances, the wetness indicators 80 may not coordinate with graphics 78. The absorbent articles 10 of the present disclosure may be free of graphics 78. Wetness indicators 80 of the present disclosure may comprise, consist essentially of, or consist of wetness indicating compositions as discussed herein. Graphics 78 may be formed from inks, pigments, colorants, and the like other than the wetness indicating compositions of the present disclosure.

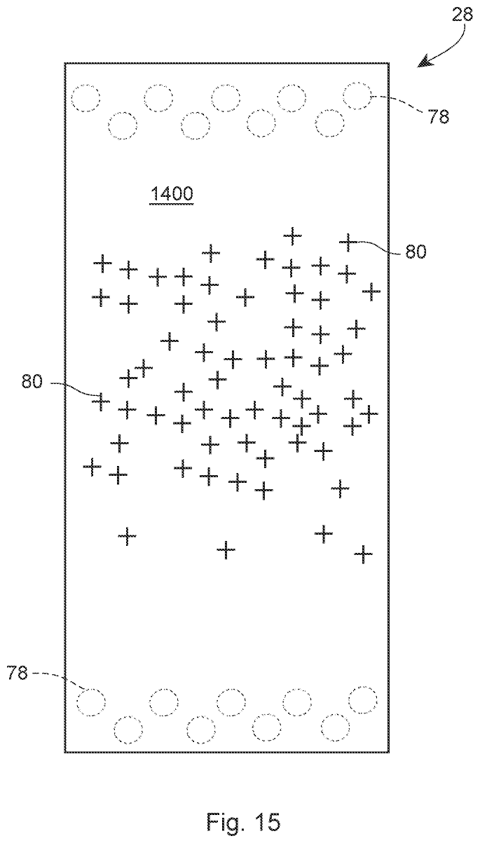

As discussed herein, the wetness indicating compositions of the present disclosure may be disposed on the absorbent article, or a component or substrate thereof, in a plurality of discrete graphics, as shown for example in FIGS. 13, 14, and 15. As shown in FIG. 14, the wetness indicators 80 may be disposed as a first plurality of discrete graphics on a first surface 1400 of the backsheet 28. As shown in FIG. 15, a graphic or graphics 78 may be disposed on a second surface of the backsheet 28, as a single graphic or as a second plurality of discrete graphics. As shown in FIG. 13, the wetness indicators 80 and a graphic or graphics 78 may be disposed on the same surface of the backsheet 28, for example a first, wearer-facing surface. The first plurality of graphics (wetness indicators 80) may not overlap the second plurality of graphics 78.

### Examples

The following examples are provided to help illustrate the wetness indicating compositions of the present disclosure. The exemplified wetness indicating compositions may be prepared by conventional formulation and mixing techniques, as discussed further herein.

EXAMPLE 1 - Printable Wetness Indicating Formulation

| Function | Chemical | Weight % |
|---|---|---|
| Colorant | Bromophenol Blue | 1-2% |
| Colorant Fastener | Tetraoctyl Ammonium Bromide | 1-4% |
| Solvent | 1-propanol | 50-65% |
| Solvent | Propyl Acetate | 5-10% |
| Solvent | Ethyl Acetate | 5-10% |
| Resin | MOWITAL BA 20S | 10-20% |
| Acid | Citric Acid | 0.2-4% |

EXAMPLE 2 - Printable Wetness Indicating Formulation

| Function | Chemical | Weight % |
|---|---|---|
| Colorant | Bromophenol Blue | 1% |
| Colorant Fastener | Tetraoctyl Ammonium Bromide | 2% |
| Solvent | 1-propanol | 65% |
| Solvent | Propyl Acetate | 8.4% |
| Solvent | Ethyl Acetate | 7.6% |
| Resin | MOWITAL BA 20S | 15% |
| Acid | Citric Acid | 1% |

EXAMPLE 3 - Printable Wetness Indicating Formulation

| Function | Chemical | Weight % |
|---|---|---|
| Colorant | Bromophenol Blue | 2% |
| Colorant Fastener | Didodecyl Dimethyl Ammonium Chloride | 4% |
| Solvent | 1-propanol | 64% |
| Solvent | Propyl Acetate | 8% |
| Solvent | Ethyl Acetate | 7% |
| Resin | MOWITAL BA 20S | 15% |

The preceding Examples 1-3 can be made by first combining the solvents in a mixing vessel. The resin can then be added to and/or dissolved in the solvent mixture via continuous mixing over time at room temperature (approx. 25° C.), whereby the time may depend upon volume and shear of the mixer. Next, the colorant, colorant fastener, and acid can be added to and/or dissolved in the solvent-resin mixture via continuous mixing over time at room temperature. Of note, the wetness indicating compositions of Examples 1-3 may be a liquid at room temperature.

In a form, the absorbent articles of the present disclosure may comprise a topsheet, a backsheet, an absorbent core disposed at least partially between the topsheet and the backsheet, and a wetness indicating composition comprising a colorant and a colorant fastener; wherein the wetness indicating composition has molar ratio of colorant fastener to colorant of between about 1:1 and about 4:1, between about 1:1 and about 2.75:1, between about 1:1 and about 2.3:1, between about 1:1 and about 2:1, or between about 1:1 and about 1.75:1; and wherein the wetness indicating composition is disposed on the absorbent article in a plurality of discrete graphics, wherein at least a portion of individual graphics of the plurality of discrete graphics has an area of between about 0.02 cm² and about 25 cm². The wetness indicating composition may comprise between about 0.3% to about 3% by weight of the wetness indicating composition of a colorant, between about 0.3% and about 3% of a colorant fastener, between about 40% and about 84.5% of a solvent, between about 10% and about 30% of a resin, between about 2% and about 8% of an acid, and between about 3% and about 15% of a viscosity modifier.

Absorbent articles comprising printable wetness indicating compositions of the present disclosure may be applied directly to at least one surface of the absorbent article backsheet by any of the printing processes disclosed herein. It is desirable for the print process to deliver enough ink over a specific area to achieve a minimal ink optical density measure as observed from the garment-facing surface of the absorbent article. The optical density measure can be obtained through any of a number of suitable optical measuring devices such as spectrophotometers; one such example of a suitable spectrophotometer is the X-RITE EXACT manufactured by X-RITE Corp. The Optical Density (OD) is a unitless measure of color saturation over a specified viewing area of the spectrophotometer lens. It can be summarized as the color intensity of a specified amount of wetness indicating composition within a specified application area. For instance, if within a 25 mm² area there is 10 mg of wetness indicating composition, that would correspond to an OD of x. If in that same area of 25 mm² there is 25 mg of wetness indicating composition of the same color and shape the OD would correspond to a value of y, where y>x numerically.

In the case of absorbent articles comprising wetness indicating compositions of the present disclosure where the total amount of printable wetness indicating composition per absorbent article is no more than 50 mg, or no more than 30 mg, or no more than about 10 mg, the optical density of the discreet graphics printed with the wetness indicating compositions of the present disclosure may be >0.4, or >0.5, or >0.8 as measured from the garment-facing surface of the absorbent article. The higher the OD value, the greater the intensity of color that is detectable by the observer, whereas, if the same total amount of printable wetness indicating composition were spread out over a larger coverage area of the backsheet the OD would be lower. The optical density at least some of the plurality of discreet graphics printed with the wetness indicating composition of the present disclosure may be >0.4, or >0.5, or >0.8 as measured from the garment-facing surface of the absorbent article when the wetness indicating composition is in a second state.

### Methods of Making Absorbent Articles Having a Wetness Indicating Composition

A substrate that may be incorporated into an absorbent article or a component of an absorbent article, such as a backsheet, may be provided. The substrate may comprise film, nonwoven, woven, foam, and combinations thereof, such as laminates of film and nonwoven. The substrate may comprise synthetic and/or natural materials, as well as processed natural materials, such as regenerated cellulose. The substrate may be translucent, transparent, semi-transparent, or opaque.

A wetness indicating composition of the present disclosure may be provided. Wetness indicating compositions of the present disclosure may be prepared by first adding a solvent or solvents to a mixing vessel. A resin, if used, may then be added to and/or dissolved in the solvent mixture under continuous mixing over time at approximately 25° C., whereby the time is dependent on the volume and shear of the mixer. Next, the colorant, colorant fastener, and acid is added to and/or dissolved in the solvent (and resin) under continuous mixing over time at approximately 25° C., whereby the time is dependent on the volume and shear of the mixer. The wetness indicating composition of the present disclosure need not be heated during preparation, which may reduce energy and equipment costs as compared to, for example, wetness indicator compositions incorporated into adhesive compositions having high melting temperatures. The resultant wetness indicating composition may be in a liquid state at 25° C.

The wetness indicating composition of the present disclosure may then be applied to the substrate by a printing process. As discussed herein, the wetness indicating composition of the present disclosure may be applied to an absorbent article or a component or substrate thereof by a flexographic printing process, a gravure printing process, a digital printing process, such as an ink jet printing process, and combinations thereof. In a form, the wetness indicating composition of the present disclosure may be applied to an absorbent article or a component or substrate of an absorbent article by continuous ink jet printing as an integrated step of a diaper manufacturing (converting) process. The wetness indicating composition of the present disclosure may be configured to be printed onto a substrate by a high speed flexographic process with a line speed of about 1,000 feet/minute to about 4,000 feet/minute. The wetness indicating composition of the present disclosure may be configured to be printed onto substrate by a rotogravure printing press with a line speed of about 1,000 feet/minute to about 4,000 feet/minute. The wetness indicating composition of the present disclosure may be configured to be printed onto a substrate by a digital printing process, such as a continuous ink jet printing process. In another form, the wetness indicating composition of the present disclosure may be applied to an absorbent article or a component thereof by a slot coating process.

The wetness indicating composition of the present disclosure may be applied to the substrate at a transfer rate of between about 1 BCM/in² and about 10 BCM/in², between about 2 BCM/in² and about 8 BCM/in², or between about 3 BCM/in² and about 10 BCM/in². The application rate of the wetness indicating composition to the substrate may result in finished absorbent articles comprising no more than 50 mg, no more than 35 mg, no more than 25 mg, no more than 15 mg, or no more than 10 mg of wetness indicating composition per absorbent article.

After application of the wetness indicating composition of the present disclosure to the substrate, the solvent portion of the wetness indicating composition may be allowed to evaporate prior to incorporating the substrate into the absorbent article.

In a form, a method of making an absorbent article comprising a wetness indicating composition of the present disclosure may comprise the steps of: providing a substrate; providing a wetness indicating composition comprising: a colorant; a solvent; and a colorant fastener; wherein the colorant has a first color state, wherein the first color state is associated with a first state of the wetness indicating composition, and wherein the wetness indicating composition is in the first state prior to exposure of the wetness indicating composition to an activating liquid; wherein the colorant has a second, different color state, wherein the second color state is associated with a second state of the wetness indicating composition, and wherein the wetness indicating composition is in the second state after exposure to the activating liquid; wherein the wetness indicating composition has molar ratio of colorant fastener to colorant of between about 1:1 and about 3:1, between about 1:1 and about 2.65:1, between about 1:1 and about 2.3:1, between about 1:1 and about 2:1, or between about 1:1 and about 1.75:1; and wherein the wetness indicating composition is in a liquid state at 25° C.; and applying the wetness indicating composition to the substrate by a printing process; and joining the substrate to at least one other component to form the absorbent article.

### Combinations of the Disclosure

The following combinations are provided as examples in accordance with the disclosure herein and are not intended to limit the scope of the disclosure.
1. An absorbent article comprising: a topsheet; a backsheet; an absorbent core disposed at least partially between the topsheet and the backsheet; and a wetness indicating composition comprising a colorant and a colorant fastener; wherein the colorant has a first color state, wherein the first color state is associated with a first state of the wetness indicating composition; wherein the colorant has a second, different color state, wherein the second color state is associated with a second state of the wetness indicating composition; and wherein the wetness indicating composition is disposed on the absorbent article in a plurality of discrete graphics, wherein at least a portion of individual graphics of the plurality of discrete graphics has an area of between about 0.02 cm² and about 25 cm², between about 0.035 cm² and about 15 cm², between about 0.05 cm² and about 10 cm², between about 0.05 cm² and about 5 cm², between about 0.065 cm² and about 2.5 cm², or between about 0.065 cm² and about 1.0 cm².
2. The absorbent article of paragraph 1, wherein the wetness indicating composition is in the first state prior to exposure of the wetness indicating composition to an activating liquid.
3. The absorbent article of any of the preceding paragraphs, wherein the wetness indicating composition is in the second state after exposure of the wetness indicating composition to an activating liquid.
4. The absorbent article of any of paragraphs 2-3, wherein the activating liquid is urine.
5. The absorbent article of any of paragraphs 2-4, wherein the wetness indicating composition is substantially insoluble in the activating liquid.
6. The absorbent article of any of the preceding paragraphs, wherein the colorant comprises sulfonephthalein.
7. The absorbent article of any of the preceding paragraphs, wherein the colorant comprises bromocresol green.
8. The absorbent article of any of paragraphs 1-6, wherein the colorant comprises bromophenol blue.
9. The absorbent article of any of the preceding paragraphs comprising no more than 50 mg, no more than 35 mg, no more than 25 mg, no more than 15 mg, or no more than 10 mg of the wetness indicating composition.
10. The absorbent article of any of the preceding paragraphs, wherein the wetness indicating composition is disposed only on a first surface of the backsheet.
11. The absorbent article of paragraph 10, wherein the wetness indicating composition is disposed on between about 0.5% and about 50%, between about 2% and about 45%, between about 5% and about 40%, or between about 8% and about 38% of the first surface of the backsheet.
12. The absorbent article of any of the preceding paragraphs, wherein the wetness indicating composition further comprises a resin, wherein the resin has a Glass Transition Temperature (Tg) of less than 150° C., preferably less than 100° C.
13. The absorbent article of any of the preceding paragraphs, wherein at least a portion of each of the plurality of discrete graphics is spaced apart from others of the plurality of discrete graphics by at least 2 mm, 5 mm, 8 mm, 12 mm, or 15 mm.
14. An absorbent article comprising: a topsheet; a backsheet comprising a garment-facing surface and a wearer-facing surface; an absorbent core disposed at least partially between the topsheet and the backsheet; and a wetness indicating composition comprising a colorant and a colorant fastener; wherein the colorant has a first color state, wherein the first color state is associated with a first state of the wetness indicating composition in which the colorant and the colorant fastener are not chemically bonded, and wherein the wetness indicating composition is in the first state prior to exposure of the wetness indicating composition to an activating liquid; wherein the colorant has a second color state, wherein the second color state is associated with a second state of the wetness indicating composition in which at least a portion of the colorant is chemically bonded to at least a portion of the colorant fastener, and wherein the wetness indicating composition is in the second state after exposure to the activating liquid; and wherein the wetness indicating composition is substantially insoluble in the activating liquid when in the second state.
15. The absorbent article of paragraph 14, wherein the activating liquid is urine.
16. The absorbent article of any of paragraphs 14 and 15, wherein the wetness indicating composition is disposed on the wearer-facing surface of the backsheet in a first plurality of discrete graphics.
17. The absorbent article paragraph 16, wherein a second plurality of discrete graphics is disposed on the wearer-facing surface and/or the garment-facing surface of the backsheet.
18. The absorbent article of paragraph 17, wherein the second plurality of discrete graphics are formed from a composition different than the wetness indicating composition.
19. The absorbent article of any of paragraphs 17-18, wherein the first plurality of discrete graphics and the second plurality of discrete graphics do not overlap.
20. The absorbent article of any of paragraphs 16-19, wherein at least a portion of each of the plurality of discrete graphics is spaced apart from others of the plurality of discrete graphics by at least 2 mm, 5 mm, 8 mm, 12 mm, or 15 mm.
21. The absorbent article of any of paragraphs 16-20, wherein at least a portion of individual graphics of the plurality of discrete graphics has an area of between about 0.02 cm² and about 25 cm², between about 0.035 cm² and about 15 cm², between about 0.05 cm² and about 10 cm², between about 0.05 cm² and about 5 cm², between about 0.065 cm² and about 2.5 cm², or between about 0.065 cm² and about 1.0 cm².
22. The absorbent article of any of paragraphs 14-21, wherein the wetness indicating composition is disposed on between about 0.5% and about 50%, between about 2% and about 45%, between about 5% and about 40%, or between about 8% and about 38% of the wearer-facing surface of the backsheet.
23. The absorbent article of any of paragraphs 14-22, wherein the wetness indicating composition further comprises a resin.
24. The absorbent article of any of paragraphs 14-23, wherein the absorbent article comprises no more than 50 mg, no more than 35 mg, no more than 25 mg, no more than 15 mg, or no more than 10 mg of wetness indicating composition.
25. The absorbent article of any of the preceding paragraphs, wherein the colorant fastener comprises or consists of a quaternary ammonium compound.
26. The absorbent article of paragraph 25, wherein the colorant fastener comprises or consists of a quaternary ammonium compound selected from the group consisting of: dialkyl dimethyl ammonium quaternary compounds; alkyl trimethyl ammonium chloride compounds; imidazoline quaternary compounds; mono-, di, tri-, and tetra-amidoamine quaternary compounds; mono-, di-, tri-, and tetra-benzyl quaternary compounds; benzylalkyl quaternary compounds; cationic diquaternary compounds; cationic ethoxylated quaternary compounds; mono-, di-, tri, and tetra-alkyl quaternary ammonium compounds, and combinations thereof.
27. The absorbent article of any of the preceding paragraphs, wherein the plurality of discrete graphics comprises at least 10 individual graphics.
M1. A method of making an absorbent article comprising a wetness indicating composition comprising the steps of: providing a substrate; providing a wetness indicating composition comprising: a colorant; a solvent; and a colorant fastener; wherein the colorant has a first color state, wherein the first color state is associated with a first state of the wetness indicating composition, and wherein the wetness indicating composition is in the first state prior to exposure of the wetness indicating composition to an activating liquid; wherein the colorant has a second, different color state, wherein the second color state is associated with a second state of the wetness indicating composition, and wherein the wetness indicating composition is in the second state after exposure to the activating liquid; and wherein the wetness indicating composition is in a liquid state at 25° C.; and applying the wetness indicating composition to the substrate by a printing process; and joining the substrate to at least one other component to form the absorbent article.
M2. The method of paragraph M1, wherein the wetness indicating composition is applied to the substrate by a flexographic printing process.
M3. The method of paragraph M1, wherein the wetness indicating composition is applied to the substrate by a gravure printing process.
M4. The method of paragraph M1, wherein the wetness indicating composition is applied to the substrate by a digital printing process.
M5. The method of any of paragraphs M1-M4, wherein the wetness indicating composition has a viscosity of less than 200 cP, preferably less than 100 cP, preferably less than 75 cP, as measured at 25 C.
M6. The method of any of paragraphs M1-M5, wherein the wetness indicating composition is substantially insoluble in the activating liquid when the wetness indicating composition is in the second state.
M7. The method of any of paragraphs M1-M6, wherein the activating liquid is urine.
M8. The method of any of paragraphs M1-M7, wherein the wetness indicating composition is applied to the substrate at a transfer rate of between about 1 BCM/in² and about 10 BCM/in², between about 2 BCM/in² and about 8 BCM/in², or between about 3 BCM/in² and about 10 BCM/in².
M9. The method of any of paragraphs M1-M8, wherein the wetness indicating composition is applied to the substrate in an amount of no more than 50 mg, no more than 35 mg, no more than 25 mg, no more than 15 mg, or no more than 10 mg of wetness indicating composition.
M10. The method of any of paragraphs M1-M9, wherein the wetness indicating composition is applied to the substrate in a plurality of discrete graphics, wherein at least a portion of individual graphics of the plurality of discrete graphics has an area of between about 0.02 cm² and about 25 cm², between about 0.035 cm² and about 15 cm², between about 0.05 cm² and about 10 cm², between about 0.05 cm² and about 5 cm², or between about 0.05 cm² and about 2.5 cm²
M11. The method of any of paragraphs M1-M10, wherein the wetness indicating composition is disposed on between about 0.5% and about 50%, between about 2% and about 45%, between about 5% and about 40%, or between about 8% and about 38% of a surface of the substrate.
M12. The method of any of paragraphs M1-M11, wherein the wetness indicating composition further comprises a resin.
M13. The method of paragraph M12, wherein the resin has a Glass Transition Temperature (Tg) of less than 150° C., preferably less than 100° C.
M14. The method of any of paragraphs M1-M13, wherein the solvent comprises a volatile solvent.
M15. The method of paragraph M14, wherein the solvent comprises at least one of: ethyl acetate, propyl acetate, and 1-propanal.
M16. The method of any of paragraphs M1-M14, wherein the solvent consists of ethyl acetate, propyl acetate, and 1-propanal.
M17. The method of any of paragraphs M1-M16, wherein at least a portion of each of the plurality of discrete graphics is spaced apart from others of the plurality of discrete graphics by at least 2 mm, 5 mm, 8 mm, 12 mm, or 15 mm.
M18. The method of any of paragraphs M1-M17, further comprising the step of evaporating the solvent after applying the wetness indicating composition to the substrate.
M19. The method of any of paragraphs M1-M18, wherein the colorant is not chemically bonded to the colorant fastener in the first state, and wherein at least a portion of the colorant is chemically bonded to at least a portion of the colorant fastener in the second state.
M20. The method of paragraph M19, wherein the wetness indicating composition, when in the second state, is substantially insoluble in the activating liquid.
M21. The method of any of paragraphs M1-M20, wherein the colorant fastener comprises or consists of a quaternary ammonium compound.
M22. The method of paragraph M21, wherein the colorant fastener comprises or consists of a quaternary ammonium compound selected from the group consisting of: dialkyl dimethyl ammonium quaternary compounds; alkyl trimethyl ammonium chloride compounds; imidazoline quaternary compounds; mono-, di, tri-, and tetra-amidoamine quaternary compounds; mono-, di-, tri-, and tetra-benzyl quaternary compounds; benzylalkyl quaternary compounds; cationic diquaternary compounds; cationic ethoxylated quaternary compounds; mono-, di-, tri, and tetra-alkyl quaternary ammonium compounds, and combinations thereof.
M23. The method of any of paragraphs M1-M22, wherein the wetness indicating composition has a Glass Transition Temperature (Tg) of less than 150° C., preferably less than 100° C.
M24. A method of making an absorbent article utilizing an in-line absorbent article assembly line converter comprising the steps of: providing a substrate; providing a wetness indicating composition comprising: a colorant; a solvent; and a colorant fastener; wherein the colorant has a first color state, wherein the first color state is associated with a first state of the wetness indicating composition, and wherein the wetness indicating composition is in the first state prior to exposure of the wetness indicating composition to an activating liquid; wherein the colorant has a second, different color state, wherein the second color state is associated with a second state of the wetness indicating composition, and wherein the wetness indicating composition is in the second state after exposure to the activating liquid; and wherein the wetness indicating composition is in a liquid state at 25° C.; and applying the wetness indicating composition to the substrate by a continuous ink jet printing process as a plurality of discrete graphics; and joining the substrate to at least one other component to form the absorbent article.
M25. An absorbent article made by the method of any of paragraphs M1-M24.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. An absorbent article comprising:
a topsheet;
a backsheet;
an absorbent core disposed at least partially between the topsheet and the backsheet; and
a wetness indicating composition comprising a colorant and a colorant fastener;
wherein the colorant has a first color state, wherein the first color state is associated with a first state of the wetness indicating composition;
wherein the colorant has a second, different color state, wherein the second color state is associated with a second state of the wetness indicating composition; and
wherein the wetness indicating composition is disposed on the absorbent article in a plurality of discrete graphics, wherein at least a portion of individual graphics of the plurality of discrete graphics has an area of between about 0.02 cm² and about 25 cm².

2. The absorbent article of claim 1, wherein the wetness indicating composition is in the first state prior to exposure of the wetness indicating composition to an activating liquid.

3. The absorbent article of claim 2, wherein the wetness indicating composition is in the second state after exposure of the wetness indicating composition to an activating liquid.

4. The absorbent article of claim 3, wherein the wetness indicating composition when in the second state is substantially insoluble in the activating liquid.

5. The absorbent article of claim 1, wherein the colorant comprises sulfonephthalein.

6. The absorbent article of claim 1, wherein the colorant comprises bromocresol green.

7. The absorbent article of claim 1, wherein the colorant comprises bromophenol blue.

8. The absorbent article of claim 1 comprising no more than 50 mg of the wetness indicating composition.

9. The absorbent article of claim 1, wherein the wetness indicating composition is disposed only on a first surface of the backsheet.

10. The absorbent article of claim 9, wherein the wetness indicating composition is disposed on between about 0.5% and about 50% of the first surface of the backsheet.

11. The absorbent article of claim 1, wherein the wetness indicating composition further comprises a resin.

12. The absorbent article of claim 11, wherein the resin has a Glass Transition Temperature (Tg) of less than 150° C.

13. The absorbent article of claim 1, wherein the wetness indicating composition has a viscosity of less than 200 cP as measured at 25° C.

14. The absorbent article of claim 1, wherein at least a portion of each of the plurality of discrete graphics is spaced apart from others of the plurality of discrete graphics by at least 2 mm.

15. An absorbent article comprising:
a topsheet;
a backsheet comprising a garment-facing surface and a wearer-facing surface;
an absorbent core disposed at least partially between the topsheet and the backsheet; and
a wetness indicating composition comprising a colorant and a colorant fastener;
wherein the colorant has a first color state, wherein the first color state is associated with a first state of the wetness indicating composition in which the colorant and the colorant fastener are not chemically bonded, and wherein the wetness indicating composition is in the first state prior to exposure of the wetness indicating composition to an activating liquid;
wherein the colorant has a second color state, wherein the second color state is associated with a second state of the wetness indicating composition in which at least a portion of the colorant is chemically bonded to at least a portion of the colorant fastener, and wherein the wetness indicating composition is in the second state after exposure to the activating liquid;
wherein the wetness indicating composition is substantially insoluble in the activating liquid when in the second state; and
wherein the wetness indicating composition is disposed on the absorbent article in a first plurality of discrete graphics.

16. The absorbent article of claim 15, wherein the first plurality of discrete graphics is disposed on the wearer-facing surface of the backsheet.

17. The absorbent article claim 16, wherein a second plurality of discrete graphics is disposed on the wearer-facing surface and/or the garment-facing surface of the backsheet.

18. The absorbent article of claim 17, wherein the second plurality of discrete graphics are formed from a composition different than the wetness indicating composition.

19. The absorbent article of claim 15, wherein each graphic of the first plurality of discrete graphics is spaced apart from others of the first plurality of discrete graphics by at least 2 mm.

20. The absorbent article of claim 15, wherein at least a portion of each graphic of the first plurality of discrete graphics has an area of between about 0.02 cm² and about 25 cm².

21. The absorbent article of claim 15, wherein the wetness indicating composition is disposed on between about 0.5% and about 50% of the wearer-facing surface of the backsheet.

22. The absorbent article of claim 15, wherein the wetness indicating composition further comprises a resin.

23. The absorbent article of claim 22, wherein the resin has a Glass Transition Temperature (Tg) of less than 150° C.

24. The absorbent article of claim 15, wherein the absorbent article comprises no more than 50 mg of wetness indicating composition.
